(19) United States
(12) Patent Application Publication
(10) Pub. No.: US 2026/0331223 A1
(43) Pub. Date: Oct. 22, 2026

(54) MACHINE LEARNING TECHNIQUES FOR TRUNCATION PREDICTION IN AAV DESIGNS

(71) Applicant: University of Massachusetts, Westborough, MA (US)

(72) Inventors: Guangping Gao, Worcester, MA (US); Phillip Tai, Worcester, MA (US); Sandhiya Ravi, Worcester, MA (US); Mengtian Cui, Worcester, MA (US)

(73) Assignee: University of Massachusetts, Westborough, MA (US)

(21) Appl. No.: 19/638,244

(22) Filed: Apr. 3, 2026

Related U.S. Application Data
(60) Provisional application No. 63/787,226, filed on Apr. 11, 2025.

(51) Int. Cl. G16B 40/00; C12N 15/86

(52) U.S. Cl. CPC G16B 40/00; C12N 15/86; C12N 2750/14143

(57) ABSTRACT

Aspects of the disclosure relate to computational techniques to predict truncation events in recombinant adeno-associated virus (rAAV) vectors. The computational techniques include a method, a system, and at least one non-transitory computer-readable storage medium for performing: obtaining rAAV vector sequence data and processing the rAAV vector sequence data using a trained neural network model to obtain an output indicating one or more positions in the rAAV vector sequence likely to be locations of truncation events. The trained neural network model may include a series of convolutional layers followed by a recurrent layer configured to process output of the series of convolutional layers and to generate the output indicating the one or more positions in the rAAV vector sequence likely to be locations of truncation events.

### RELATED APPLICATIONS

This Application claims the benefit under 35 USC 119(e) of U.S. Provisional Application Ser. No. 63/787,226, filed Apr. 11, 2025, entitled “MACHINE LEARNING TECHNIQUES FOR TRUNCATION PREDICTION IN AAV DESIGNS”, the entire disclosure of which is hereby incorporated by reference in its entireties.

### BACKGROUND

Recombinant adeno-associated virus (rAAV) vectors are imperfect. As such, essential components to the development of rAAV vectors for gene therapy are efficacy, safety, and cost-effective production of vectors. Developing effective recombinant rAAV vectors is especially challenging because occurrence of truncation events during vector genome replication and packaging leads to dysfunctional vectors. That is, the truncation events compromise the efficacy of gene therapy.

### SUMMARY

Some embodiments provide for a method for using machine learning to predict truncation events in recombinant adeno-associated virus (rAAV) vectors. The method comprises using at least one computer hardware processor to perform: obtaining rAAV vector sequence data; and processing the rAAV vector sequence data using a trained neural network model to obtain an output indicating one or more positions in the rAAV vector sequence likely to be locations of truncation events. The trained neural network model comprises a series of convolutional layers followed by a recurrent layer configured to process output of the series of convolutional layers and to generate the output indicating the one or more positions in the rAAV vector sequence likely to be locations of truncation events.

Some embodiments provide for a method for selecting a particular rAAV vector sequence from a plurality of rAAV vector sequences, the method comprising: performing the method for predicting truncation events as described herein on each of the plurality of rAAV vector sequences to obtain a respective plurality of outputs characterizing predicted truncation behavior of the plurality of rAAV vector sequences; and selecting the particular rAAV vector sequence based on the plurality of outputs characterizing the predicted truncation behavior of the plurality of rAAV vector sequences.

Some embodiments provide for a system, comprising: at least one computer hardware processor; and at least one non-transitory computer-readable storage medium storing processor-executable instructions that, when executed by the at least one computer hardware processor, cause the at least one computer hardware processor to perform a method for using machine learning to predict truncation events in recombinant adeno-associated virus (rAAV) vectors. The method comprises using at least one computer hardware processor to perform: obtaining rAAV vector sequence data; and processing the rAAV vector sequence data using a trained neural network model to obtain an output indicating one or more positions in the rAAV vector sequence likely to be locations of truncation events. The trained neural network model comprises a series of convolutional layers followed by a recurrent layer configured to process output of the series of convolutional layers and to generate the output indicating the one or more positions in the rAAV vector sequence likely to be locations of truncation events.

Some embodiments provide for a system, comprising: at least one computer hardware processor; and at least one non-transitory computer-readable storage medium storing processor-executable instructions that, when executed by the at least one computer hardware processor, cause the at least one computer hardware processor to perform a method for selecting a particular rAAV vector sequence from a plurality of rAAV vector sequences, the method comprising: performing the method for predicting truncation events as described herein on each of the plurality of rAAV vector sequences to obtain a respective plurality of outputs characterizing predicted truncation behavior of the plurality of rAAV vector sequences; and selecting the particular rAAV vector sequence based on the plurality of outputs characterizing the predicted truncation behavior of the plurality of rAAV vector sequences.

Some embodiments provide for at least one non-transitory computer-readable storage medium storing processor-executable instructions that, when executed by at least one computer hardware processor, cause the at least one computer hardware processor to perform a method for using machine learning to predict truncation events in recombinant adeno-associated virus (rAAV) vectors. The method comprises using at least one computer hardware processor to perform: obtaining rAAV vector sequence data; and processing the rAAV vector sequence data using a trained neural network model to obtain an output indicating one or more positions in the rAAV vector sequence likely to be locations of truncation events. The trained neural network model comprises a series of convolutional layers followed by a recurrent layer configured to process output of the series of convolutional layers and to generate the output indicating the one or more positions in the rAAV vector sequence likely to be locations of truncation events.

Some embodiments provide for at least one non-transitory computer-readable storage medium storing processor-executable instructions that, when executed by at least one computer hardware processor, cause the at least one computer hardware processor to perform a method for selecting a particular rAAV vector sequence from a plurality of rAAV vector sequences, the method comprising: performing the method for predicting truncation events as described herein on each of the plurality of rAAV vector sequences to obtain a respective plurality of outputs characterizing predicted truncation behavior of the plurality of rAAV vector sequences; and selecting the particular rAAV vector sequence based on the plurality of outputs characterizing the predicted truncation behavior of the plurality of rAAV vector sequences.

Some embodiments provide for a recombinant adeno-associated virus (rAAV) comprising: (i) an rAAV vector comprising a sequence processed according to the method for predicting truncation events as described herein; and (ii) one or more AAV capsid proteins.

Some embodiments provide for a composition comprising: (i) a population of recombinant adeno-associated virus (rAAV) particles comprising less than 10% truncated rAAV vector genomes; and (ii) mammalian cell culture media.

In some embodiments, the series of convolutional layers comprises a series of one-dimensional (1D) layers.

In some embodiments, the series of 1D convolutional layers comprise filters of increasing depth.

In some embodiments, the series of 1D convolutional layers identify sequence motifs linked to truncation-prone regions of the rAAV vector sequence.

In some embodiments, the trained neural network model comprises maximum pooling layers within the series of convolutional layers.

In some embodiments, the recurrent layer comprises a series of gated recurrent units (GRUs).

In some embodiments, the series of GRUs comprises a series of bi-directional GRUs (Bi-GRUS).

In some embodiments, the recurrent layer comprises a long-short term memory (LSTM) layer.

In some embodiments, the trained neural network model further comprises a classification head configured to process output of the recurrent layer to generate output comprising: (1) an indication of whether or the rAAV vector sequence is truncation prone; and/or (2) an estimated expected frequency of truncation events for the rAAV vector sequence.

In some embodiments, the classification head comprises one or more fully-connected layers.

In some embodiments, the method further comprises synthesizing the particular rAAV vector sequence.

In some embodiments, the synthesizing comprises solid-phase oligonucleotide synthesis.

In some embodiments, the synthesized particular rAAV vector sequence is comprised in a plasmid.

In some embodiments, the method further comprises introducing the synthesized particular rAAV vector sequence into a mammalian cell.

In some embodiments, the mammalian cell is configured to express one or more AAV capsid proteins, one or more AAV Rep proteins, and/or one or more Adenoviral helper proteins.

In some embodiments, the mammalian cell is a human embryonic kidney 293 (HEK293) cell.

In some embodiments, the rAAV vector sequence comprises two wild-type AAV inverted termina repeats (ITRs).

In some embodiments, the wild-type AAV ITRs are AAV2 ITRs.

In some embodiments, the rAAV vector sequence comprises at least one mutant AAV inverted terminal repeat (ITR).

In some embodiments, the at least one mutant ITR is a deltaITR.

The foregoing summary is a non-limiting summary of the invention, which is defined by the attached claims.

### BRIEF DESCRIPTION OF DRAWINGS

Various aspects and embodiments will be described with reference to the following figures. It should be appreciated that the figures are not necessarily drawn to scale. Items appearing in multiple figures are indicated by the same or a similar reference number in all the figures in which they appear.

FIG. 1 shows a block diagram of an example system for predicting truncation events, in accordance with some embodiments of the technology described herein.

FIG. 2 shows a flow diagram for an example process of using machine learning to predict truncation events in rAAV vectors in accordance with some embodiments of the technology described herein.

FIG. 3 shows a flow diagram for an example process of selecting a particular rAAV vector sequence from a plurality of rAAV vector sequences in accordance with some embodiments of the technology described herein.

FIG. 4 shows exemplary architecture of the machine learning model for predicting truncation events in rAAV vectors in accordance with some embodiments of the technology described herein.

FIG. 5 shows predicted vs. actual read counts across a first test rAAV construct in the forward sequence (left) and reverse complement sequence (right) in accordance with some embodiments of the technology described herein. The bottom panels compare actual read counts (blue) with model-predicted counts (pink) across genome positions, demonstrating the model's ability to accurately predict truncation events within the vector genome.

FIG. 6 shows predicted vs. actual read counts across a second test rAAV construct in the forward sequence (left) and reverse complement sequence (right) in accordance with some embodiments of the technology described herein. The bottom panels compare actual read counts (blue) with model-predicted counts (pink) across genome positions, demonstrating the model's ability to accurately predict truncation events within the vector genome.

FIGS. 7A-7C show performance of the trained machine learning model, including (FIG. 7A) loss curves, (FIG. 7B) confusion matrix, and (FIG. 7C) table summary of precision, recall, F-1 score, and support values in accordance with some embodiments of the technology described herein.

FIG. 8 is a block diagram of an illustrative computer system that may be used in implementing some embodiments of the technology described herein.

FIG. 9 is a schematic depicting model-guided stratification of random sequences based on predicted rAAV truncation risk enables experimental validation. Randomly generated DNA sequences are first evaluated using the trained deep learning model and stratified into high, medium, low, or null truncation risk categories based on predicted truncation propensity. Representative sequences from each category are inserted into rAAV vector genomes at a defined genomic position and packaged into AAV9 particles. Following vector production, encapsidated genomes are isolated, assessed by alkaline gel electrophoresis, and subjected to single-molecule real-time (SMRT) long-read sequencing. This strategy enables direct experimental testing of model predictions and demonstrates the application of predictive modeling to rational rAAV genome design.

FIG. 10 shows representative data for experimental validation of predicted truncation risk by alkaline gel electrophoresis. Alkaline gel electrophoresis analysis of rAAV genomes containing model-selected sequence inserts stratified by predicted truncation risk. Random sequences classified by the deep learning model as high, medium, low, or null truncation potential were inserted into rAAV vector genomes and packaged into AAV9 particles. Encapsidated vector DNA was isolated and analyzed under denaturing (alkaline) conditions to resolve full-length and truncated genome species. Lanes corresponding to high-and medium-risk sequences display prominent truncated DNA fragments at discrete sizes (~2.0-3.0 kb), in addition to full-length genomes (~5.0 kb). In contrast, vectors containing low-and null-risk sequences predominantly exhibit full-length genome bands with markedly reduced truncated species. Molecular weight markers (M) and positive control (POS) lanes are shown for reference. These results experimentally validate the model's ability to stratify sequences by truncation susceptibility and demonstrate that predicted risk correlates with observable truncation outcomes during rAAV production.

### DETAILED DESCRIPTION

Gene therapy aims to treat or improve diseases and conditions by producing a therapeutic effect through alterations of genetic materials of a patient's cells. One way of achieving gene therapy is through adeno-associated viruses (AAVs). For example, rAAVs can deliver a functioning gene into a patient's cells to restore deficiencies, which in turn can improve the underlying disease.

Trial-and-error is a traditional way of designing rAAV vectors for gene therapy. During vector genome replication and packaging, truncation events occur, and they often lead to dysfunctional vectors. The packaged AAV genomes are not as uniform as expected, and encapsulated vector genomes may include vectors of varying lengths, host cell DNA, plasmid backbones, and recombinant contaminants. Thus, there is a need for predicting truncation events at an early stage, prior to the vectors' being manufactured and packaged. Such technology will improve vector potency and safety and reduce time and cost burdens associated with the rAAV development for gene therapy.

The inventors have appreciated the need for computational techniques for predicting truncation events de novo from the rAAV vector sequence. This de novo analysis—meaning analysis of genome sequences prior to manufacturing vectors—enables preemptive designs of rAAV vectors for manufacturing, thereby ensuring the efficacy of gene therapy when compared to time-consuming and expensive conventional trial-and-error based approach to rAAV vector production. The inventors have further appreciated that a technique for selecting a particular rAAV vector sequence, e.g., for manufacturing a composition, would offer the opportunity to increase the success rate of rAAV vector-based gene therapy by enabling the design of high homogeneity and high tier vectors while saving time and cost associated with the production of vectors.

Upon recognizing the importance of predicting truncation events de novo, the inventors have developed a trained machine learning (ML) model for predicting truncation events from the rAAV vector sequence. In some embodiments, the trained ML model may be a trained neural network model. As described herein, in some embodiments, the trained ML may have an architecture combining both convolutional layer(s) (e.g., to capture short-range dependencies) and recurrent layer(s) to capture longer-range dependencies.

This approach has several advantages over conventional approaches. The first advantage is that conventional non-computational techniques of designing rAAV vectors cannot predict the truncation events de novo, prior to the production of the rAAV vectors. Besides conventional, non-computational techniques, prior computational techniques attempting to predict the truncation events still encounter major challenges because of the lack of predictive power. For example, conventional rule-based genomic analysis of rAAV vector sequence data ignores long-range dependencies that influence the likelihood of truncation events. Moreover, conventional computational techniques lack extensive training dataset or focus on particular types of rAAV constructs. Thus, they do not work well on constructs having varying lengths and varying regulatory elements.

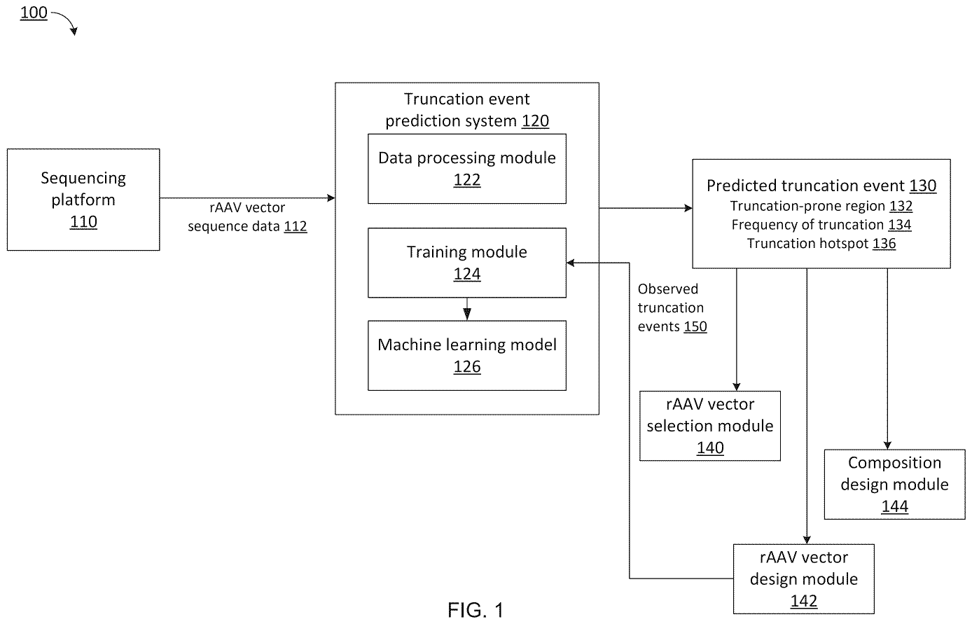

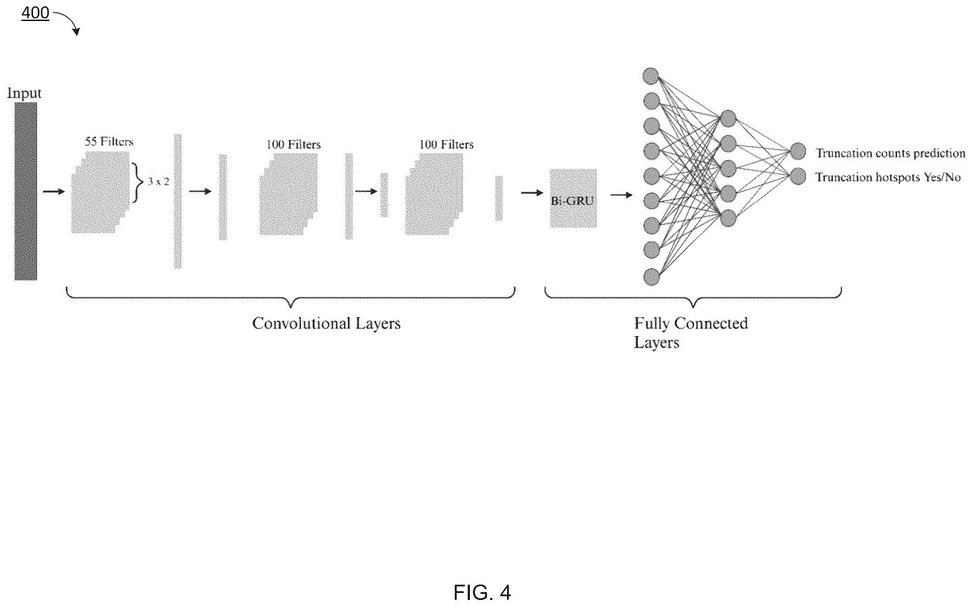

The inventors have appreciated these shortcomings of conventional techniques for predicting truncation events and have recognized that such shortcomings can be avoided by using a two-component machine learning approach, with different components aimed at capturing local and long-rage features influencing the likelihood of truncation events in rAAV vector sequences. In some embodiments, this is achieved by using a trained neural network model including one or more convolutional layers (e.g., to capture influence of local features) and one or more recurrent layers (e.g., to capture influence of long-range features), as described herein including with reference to FIG. 1 and FIG. 4.

By improving the prediction accuracy, techniques described herein addresses critical deficiencies in AAV vector manufacturing, including high failure rates due to unexpected truncation events, a limited understanding of the molecular mechanisms driving the contaminating events, and excessive costs and time associated with post-production screening. By shifting post-hoc detection of contaminating events to de novo truncation prediction and model optimization, the techniques described herein and developed by the inventors enable the development of more stable and functional rAAV vectors, reducing manufacturing inefficiencies and improving therapeutic outcomes. This approach represents a paradigm shift in AAV vector engineering, setting a new standard for efficiency, scalability, and precision in gene therapy applications.

Following below are more detailed descriptions of various concepts related to, and embodiments of, predicting truncation events in rAAV vectors using machine learning. It should be appreciated that various aspects described herein may be implemented in any of numerous ways. Examples of specific implementations are provided herein for illustrative purposes only. In addition, the various aspects described in the embodiments below may be used alone or in any combination and are not limited to the combinations explicitly described herein.

FIG. 1 shows a block diagram of an example system that uses machine learning techniques for predicting truncation events. As shown in FIG. 1, the example system 100 includes a sequencing platform 110 and a truncation event prediction system 120. The sequencing platform 110 sequences rAAV genome sequences and outputs rAAV vector sequence data 112. In some embodiments, the sequencing platform 110 uses nanopore or single molecule real-time (SMRT) sequencing technologies to ensure robustness and generalizability. In some embodiments, the rAAV vector sequence data 112 is sourced from existing dataset of rAAV vector sequences. In some embodiments, the sequencing platform 110 applies AAV-GPseq analyses of vectors, ranging from around 2.5 kb to around 4.7 kb reference genomes with different regulatory elements.

A truncation event prediction system 120 receives the rAAV vector sequence data 112 and applies one or more of its subsystems: a data processing module 122, a training module 124, and a trained machine learning model 126. The data processing module 122 processes the received rAAV vector sequence data 112. In some embodiments, the data processing module 122 is configured to filter, normalize, and/or annotate the rAAV vector sequence data 112. For example, the data processing module 122 is configured to annotate each rAAV vector sequence with experimentally observed truncation events. Such annotations allow machine learning models, during training, to learn sequence determinants associated with genomic instability.

The training module 124 is configured to train a machine learning model using training data and generate a trained machine learning model such as trained machine learning model 126. In some embodiments, the training data may be the rAAV vector sequence data 112 and/or the processed rAAV vector sequence data 112, obtained from the data processing module 122. For example, in some embodiments, the trained machine learning model 126 may be a machine learning model trained on a dataset of rAAV genome sequences, including both single-stranded (ssAAV) and self-complementary (scAAV) vectors. These sequences include AAV-GPseq vectors representing promoter, transgenes, and capsid types with reference genomes from around 2.5 kb to 4.7 kb. Promoters include CB6, RLBP1, PLE316, GUSB, hSMN, SMN, hCox20, Ch3L1, mAspaP-dCNS, and mAspaP-DNHS. Transgenes include EGFP, Fluc, KH902, hSMN-miR208-122T, native hSMN, GFAP nanobody, amiR for Ataxin02, hCox20-201 codon optimized, hCox20-203 codon optimized, hASPA, and sgRNA. Capsid types include AAV1, AAV2, AAV2.7m8, AAV3b, AAV5, AAV8, AAV9, engineered AAV2, and engineered AAV9. The extensive training dataset represents vector heterogeneity across diverse vector architectures.

In some embodiments, the trained machine learning model 126 is a trained neural network model. In some embodiments, the trained neural network model has one or more convolutional layers and one or more recurrent layers (e.g., bidirectional gated recurrent units (Bi-GRUs)) to capture both local sequence features and long-range dependencies. An example architecture for the trained machine learning model 126 is described herein in connection with FIG. 4. The training data reflects features such as genomic position, nucleotide sequence, GC content, and dG (which is helpful for prediction because sequences with hairpin-like structure, high GC content, and low dG value are more likely to be truncation hotspots (regions where the truncation events are frequently observed)). In some embodiments, the training module 124 is configured to use contextual features indicative of the long-range dependencies of the rAAV vector sequence data by a sliding window approach, e.g., 50 to 200 base pairs.

When applied to the rAAV sequence data, the trained machine learning model 126 generates the predicted truncation event 130. In some embodiments, the predicted truncation event 130 generates one or more of a truncation-prone region 132 (genomic positions of the rAAV vector sequence that is likely to have truncation events), a frequency of truncation 134 for each position in the sequence, and a truncation hotspot 136 (regions where the truncation events are frequently observed). In some embodiments, the trained machine learning model 126 applies a binary classification to identify whether a sequence, or portions of the sequence, is truncation-prone to generate the truncation-prone region 132 and the truncation hotspot 136. In some embodiments, the trained machine learning model 126 applies a regression-based quantification to estimate the frequency of truncation 134. In some embodiments, the trained machine learning model 126 uses the estimated frequency of truncation 134 to generate the truncation-prone region 132 and the truncation hotspot 136. The trained machine learning model 126's ability to generate multiple predictions is useful in detecting not only truncation-prone regions but also in providing critical insights into severity of truncation events.

For example, the predicted truncation event 130 may be represented using a binary encoding, indicating 0 for a genomic position not likely to be truncated and 1 otherwise (e.g., 0011111000). In some embodiments, the predicted truncation event 130 includes predicted amplitude of truncation events indicative of the severity of truncation events where the encoding includes a numeric indication of the amplitude. For example, the encoding “0012789000” uses the values 1, 2, 7, 8, and 9 to indicate different likelihoods of truncation events (e.g., with “8” indicating a higher likelihood of occurrence of the event than “2”).

The system 100 and trained ML model 126 can be used to guide rAAV vector selection, design, as well as a composition design.

For example, with respect to rAAV vector selection, rAAV vector selection module 140 may be configured to apply the trained machine learning model 126 to each of the plurality of rAAV vector sequences to obtain a respective plurality of predicted truncation events characterizing predicted truncation behavior of the plurality of rAAV vector sequences. In turn, these predicted truncation events may be used to select one or more rAAV vectors for subsequent use. That selection may be performed using any suitable selection criteria (e.g., selecting rAAV vectors that have no predicted truncations anywhere or not in certain regions, selecting rAAV vectors that don't have any predicted truncations having at least a threshold likelihood, etc.).

As another example, rAAV vector design module 142 may guide users in the process of rAAV vector design. For example, the rAAV vector design module 142 may be configured to display, via a graphical user interface, the rAAV vector being designed and the predicted truncation events in that sequence. As users modify the rAAV vector sequence, the system may update its prediction(s) with respect to truncation events and display the updated prediction(s) in the graphical user interface. In some embodiments, the rAAV vector design module 142 may be further configured to provide users with recommendations for how an rAAV vector sequence being designed can be modified to change (e.g., remove) predicted truncations. For example, the rAAV vector design module 142 may recommend modifications that would lead to removal of a truncation hotspot. Based on the recommendation, a user can provide, as an input to system 100, a revised rAAV vector sequence such that the system 100 can determine whether modifications led to the removal of predicted truncation hotspots.

As another example, the composition design module 144 may guide users in the process of composition design. The composition may include mammalian cell culture media and a population of rAAV particles comprising less than a certain threshold of truncated rAAV vector genomes. The composition design module 144 may be configured to provide recommendations to a user how to design the composition based on the predicted truncation events. For example, the composition design module 144 may be configured to display, on a user interface, various settings to guide the selection of the rAAV particles. In some embodiments, one of the settings is a threshold on the percentage of truncated rAAV vector genome. In some embodiments, the threshold is 10%, meaning that the rAAV particle comprises less than 10% truncated rAAV vector genomes. In some embodiments, the threshold ranges from 1% to 25%, depending on the rAAV vector sequence including its length and elements as well as a particular application of the gene therapy. Depending on the nature of genetic alterations, the tolerance for the vector heterogeneity differs. Based upon receiving the user input on the user interface, the composition design module 144 is configured to display instructions how to design the composition.

In some embodiments, the system 100 may be configured to update (e.g., fine-tune) the trained ML model 126 through additional training data to obtain an updated ML model. This may serve to improve the model's predictive accuracy. In some embodiments, the additional training data may be obtained by performing laboratory experiments. Data from such wet-lab experiments may then be used to assess and improve the trained machine learning model 126's ability to predict truncation events. For example, observed truncation events 150 from the wet-lab experiments, e.g., from a newly designed rAAV vector from the rAAV vector design module 142, may serve as feedback to the training module 124 for use training ML models for predicting truncation events. In some embodiments, the training module 124 adds the observed truncation events 150 to its training dataset and generates an updated (e.g., fine-tuned) trained machine learning model 126 to be used for analyzing the next rAAV vector sequence.

### Example Processes

Example processes are described herein. The described processes below may be performed by any suitable computing device or devices. In some embodiments, the described processes are performed by the system 100 shown in FIG. 1.

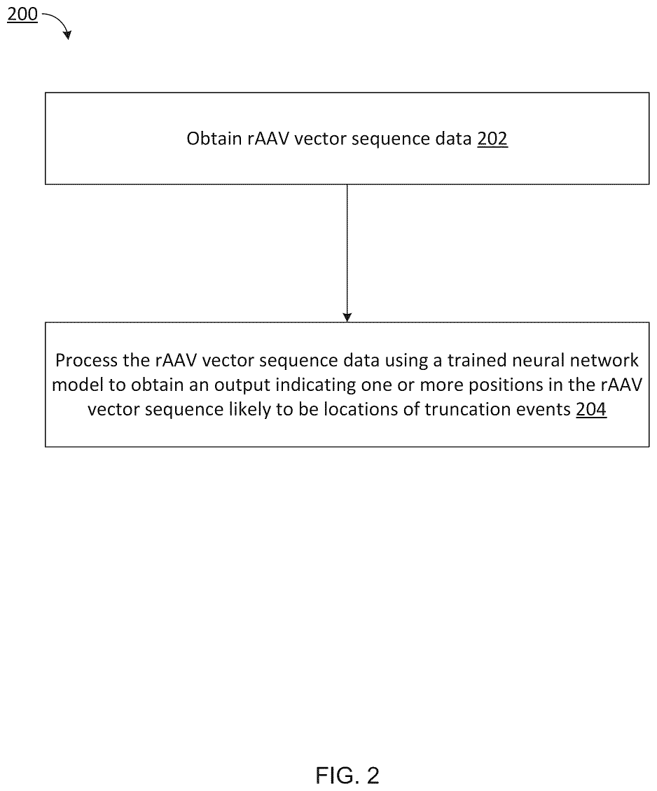

FIG. 2 shows a flow diagram for an example process 200 of using machine learning to predict truncation events in rAAV vectors. In some embodiments, the truncation event prediction system 120 shown in FIG. 1 performs the process 200.

Process 200 uses at least one computer hardware processor to: obtain rAAV vector sequence data (at 202); and process the obtained rAAV vector sequence data using a trained neural network model to obtain an output indicating one or more positions in the rAAV vector sequence likely to be locations of truncation events (at 204). The rAAV vector sequence data may represent rAAV vector genomes.

In some embodiments, the rAAV vector sequence comprises two wild-type AAV inverted termina repeats (ITRs). In some embodiments, wild-type AAV ITRs are AAV2 ITRs. In some embodiments, the rAAV vector sequence comprises at least one mutant AAV inverted terminal repeat (ITR). In some embodiments, at least one mutant ITR is a deltaITR.

In some embodiments, the system filters, normalizes, and annotates the rAAV vector sequence data. At block 204, the system uses the trained neural network model and obtains an output indicating predicted truncation events. In some embodiments, the output indicates one or more positions in the rAAV vector sequence likely to be locations of truncation events. In some embodiments, the output additionally includes truncation hotspots in the rAAV vector sequence.

In some embodiments, the trained neural network model comprises a series of one-dimensional convolutional layers followed by a recurrent layer configured to process output of the series of convolutional layers and to generate the output indicating the one or more positions in the rAAV vector sequence likely to be locations of truncation events. In some embodiments, the trained neural network model is configured to generate additional output indicating truncation hotspots in the rAAV vector sequence.

In some embodiments, the series of convolutional layers comprises a series of one-dimensional (1D) convolutional layers. In some embodiments, the series of 1D convolutional layers comprise filters of increasing depth. For example, in the example of FIG. 4, the trained neural network has a series of convolutional layers with 55, 100, and 100 filters. These convolutional layers identify sequence motifs linked to truncation-prone regions of the rAAV vector sequence. In some embodiments, the trained neural network model also comprises one or more pooling layers (e.g., maximum pooling layers) within the series of convolutional layers.

In some embodiments, the recurrent layer comprises a series of gated recurrent units (GRUs) such as a series of bi-directional GRUs (Bi-GRUs). In some embodiments, the recurrent layer comprises a long-short term memory (LSTM) layer.

In some embodiments, the trained neural network model further comprises a classification head (e.g., one or more fully-connected layers) configured to process output of the recurrent layer to generate output(s) comprising: (1) an indication of whether or the rAAV vector sequence is truncation prone; and/or (2) an estimated expected frequency of truncation events for the rAAV vector sequence. In some embodiments, the system uses this output in inferring truncation hotspots in the rAAV vector sequence.

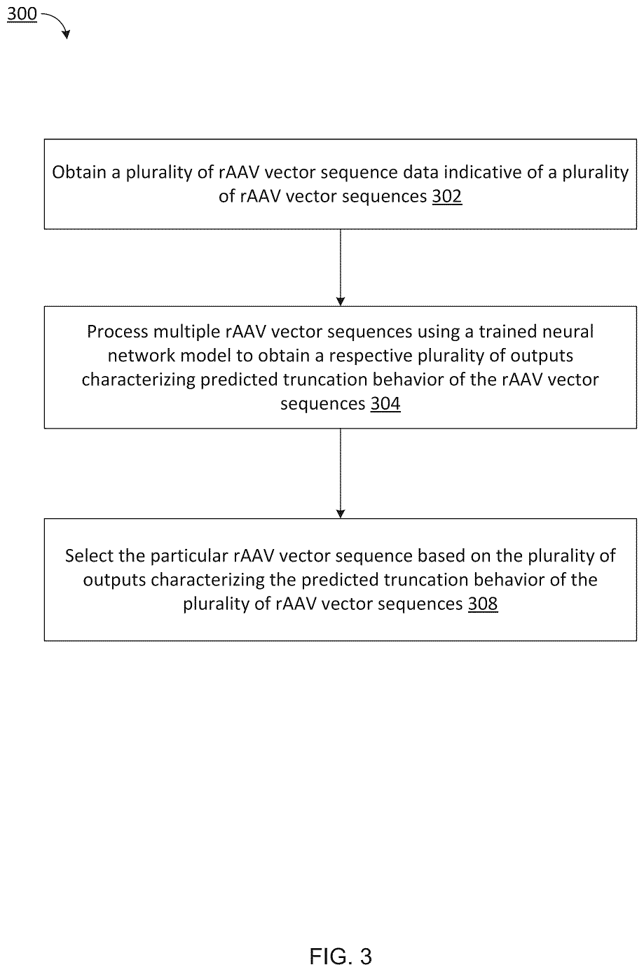

FIG. 3 shows a flow diagram for an example process 300 of selecting a particular rAAV vector sequence from a plurality of rAAV vector sequences. In some embodiments, the truncation event prediction system 120 shown in FIG. 1 performs the process 300.

Process 300 may perform the process 200 on each of multiple rAAV vector sequences and obtain a respective plurality of outputs characterizing predicted truncation behavior of the rAAV vector sequences. That is, the process 300 obtains a plurality of rAAV vector sequences (block 302) and processes the plurality of rAAV vector sequences using a trained neural network model (e.g., the trained machine learning model 126) to obtain a respective plurality of outputs characterizing predicted truncation behavior of the plurality of rAAV vector sequences (block 304). In some embodiments, the process 300 uses the trained neural network model to obtain one or more of a truncation-prone region, a frequency of truncation, and a truncation hotspot in each of the rAAV vector sequences.

At block 308, the process 300 selects the particular rAAV vector sequence based on the plurality of outputs characterizing the predicted truncation behavior of the plurality of rAAV vector sequences. In some embodiments, the process 300 selects the rAAV vector sequence with the least amount of truncation-prone regions. In some embodiments, the process 300 selects the rAAV vector sequence with the least combined frequency of truncation in the rAAV vector sequence. In some embodiments, the process 300 selects the rAAV vector sequence that meets a binary classification whether the sequence is truncation-prone. Any other suitable selection criteria may be used, as aspects of the technology described herein are not limited in this respect.

In some embodiments, the process 300 further comprises synthesizing the selected rAAV vector sequence. For example, solid-phase oligonucleotide synthesis may be used to synthesize the selected rAAV vector sequence. In some embodiments, the synthesized rAAV vector sequence is comprised in a plasmid.

In some embodiments, the process 300 may further involve introducing the synthesized rAAV vector sequence into a mammalian cell. The mammalian cell may be configured to express one or more AAV capsid proteins, one or more AAV Rep proteins, and/or one or more AAV helper proteins. In some embodiments, the mammalian cell is a human embryonic kidney 293 (HEK293) cell.

### Machine Learning Model Architecture

FIG. 4 shows example architecture of the machine learning model 400 for predicting truncation events in rAAV vectors. The ML model 400 includes an input layer that receives rAAV vector sequence data as input. The architecture 400 has a series of one-dimensional (1D) convolutional layers that extract features from the inputs. For example, the convolutional layers identify sequence motifs linked to truncation-prone regions in the sequence. The convolutional layers have filters of increasing depth, e.g., 55, 100, and 100 filters as shown in FIG. 4 with a 3×2 kernel size. After each of the convolutional layers, the architecture 400 includes a max-pooling layer to reduce dimensionality while preserving feature information. After the convolutional layers The extracted features are then processed by a recurrent layer. In some embodiments, the recurrent layer uses gated recurrent unit (GRU), e.g., bidirectional gated recurrent unit (Bi-GRU) layers. The recurrent layer captures long-range sequence dependencies, further refining the model's ability to predict truncation events. The integration of convolutional layers (for local feature extraction) and recurrent layers (for long-range sequence dependencies) allows for precise identification of truncation events. Finally, the architecture 400 has fully connected layers, which aggregate these features to generate outputs.

As shown in FIG. 4, the classification head (comprising fully connected layers and outputs) generates multiple outputs including an output (e.g., a binary output) indicative of whether a sequence is truncation-prone and a regression output (e.g., a number) indicative of the expected frequency of truncation events. Additionally, the model 400 may provide output (as output of the recurrent layer) indicating, for each of one or more particular positions in the rAAV vector sequence, whether there is likely to be a truncation event occurring at that particular position.

It should be appreciated that the neural network architecture illustrated in FIG. 4 is an example and that there are variations. For example, a different number of convolutional layers may be used, a different number of channels may be used, and/or different kernel sizes may be used. Any of various types of recurrent layers may be used (e.g., an LSTM instead of a Bi-GRU architecture). Various of types of non-linearities may be employed.

The model 400 may be trained in any suitable way. For example, gradient descent with backpropagation may be used to train the model 400. In some embodiments, to ensure precise weight updates and stable training, the model 400 may be optimized using root mean square propagation (RMSprop) with a low learning rate (e.g., 0.0001). Furthermore, the model 400 may be trained using any suitable loss function including a customized loss function, combining Huber loss for regression tasks (e.g., for estimating expected frequency of truncation events) and binary cross-entropy (weighted at, e.g.,0.25) for classification (e.g., for classifying the sequence as being truncation prone or not). To prevent overfitting and enhance generalization, the model 400 may be trained using dropout regularization (40-50%), along with adaptive learning rate adjustments, enabling stable convergence across validation datasets.

### Experimental Validation With Benchmark rAAV Constructs

The experimental validation was conducted to evaluate the predictive accuracy of the trained machine learning model by using two representative benchmark rAAV constructs (also referred to as a first test rAAV construct, “test construct 1”; and a second test rAAV construct, “test construct 2”).

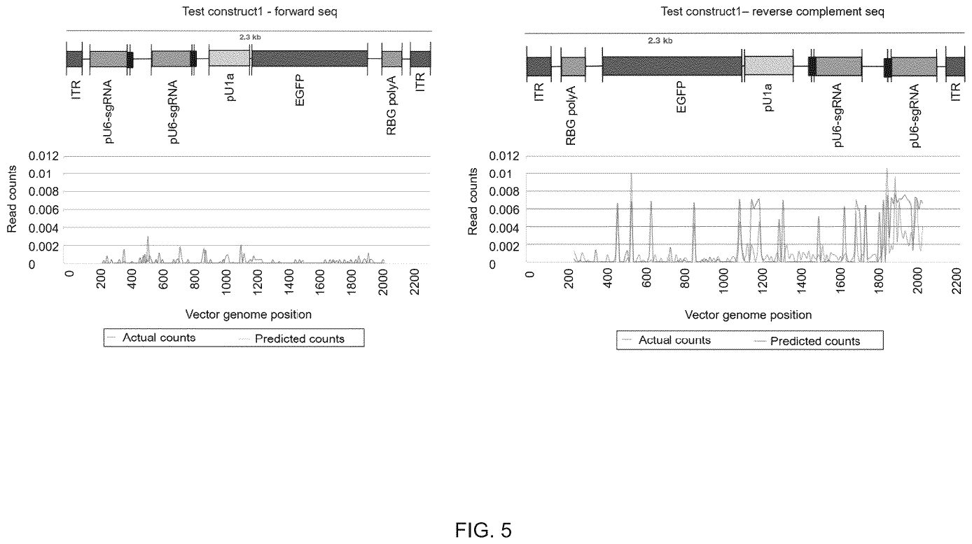

Referring to FIG. 5 (left corner), test construct 1 is a 2.3-kb sequence of U1a promoter+EGFP. Referring to FIG. 5 (right corner), test construct 2 is a 3.9-kb sequence of CMV promoter+polyA. These constructs were selected to test the trained machine learning model's ability to predict truncation events across different vector architectures with varying regulatory elements.

FIG. 5 shows predicted vs. actual read counts across a first test rAAV construct in the forward sequence (left) and reverse complement sequence (right). The actual read counts were obtained from AAV-GPseq data. Across both the forward sequence and the reverse complement sequence, there is a strong concordance between predicted and actual truncation peaks.

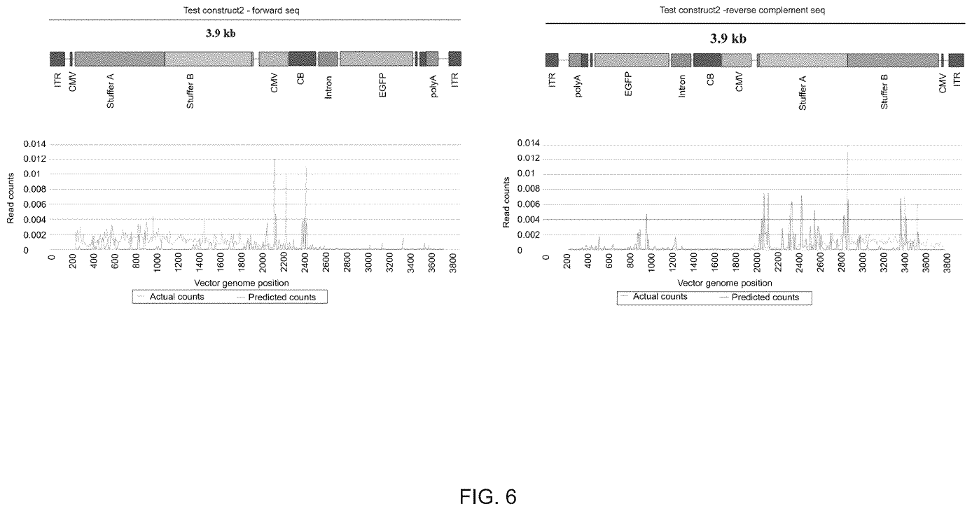

FIG. 6 shows predicted vs. actual read counts across a second test rAAV construct in the forward sequence (left) and reverse complement sequence (right). Yet again, there is a strong concordance between predicted and actual truncation peaks across both the forward sequence and the reverse complement sequence.

These results demonstrate the trained machine learning model's ability to predict truncation-prone regions before vector production, thereby reducing the need for iterative redesign. Furthermore, given the difference between the test constructs' sequence length and varying regulatory elements, the trained machine learning model is effective for a wide variety of rAAV vectors.

### Performance Metrics and Model Robustness

Model performance has been assessed using multiple metrics to validate the machine learning model's classification and regression predictive powers.

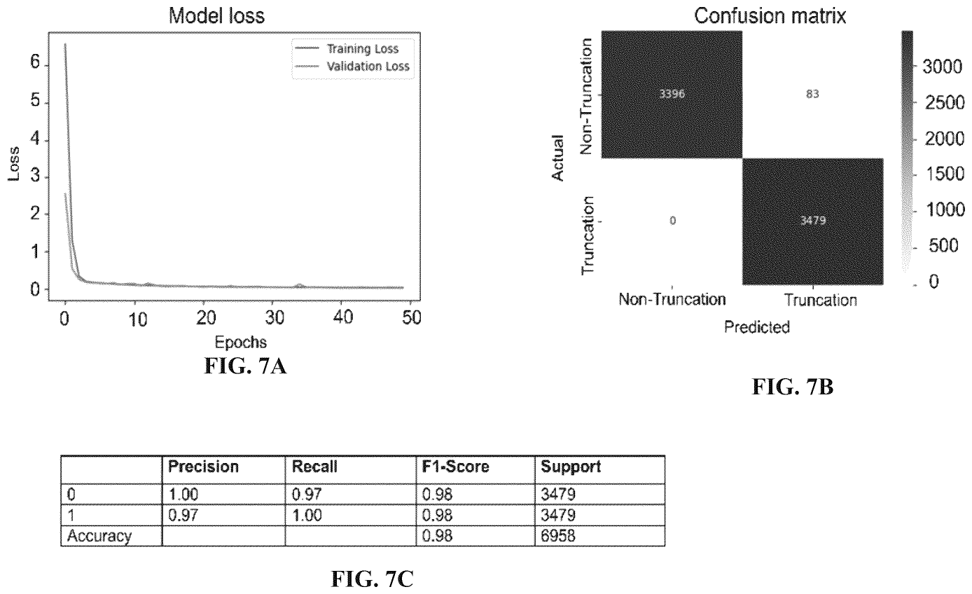

FIGS. 7A-7C show performance of the machine learning model, including (FIG. 7A) loss curves, (FIG. 7B) confusion matrix, and (FIG. 7C) table summary of precision, recall, F-1 score, and support values.

FIG. 7A shows loss curves over multiple training epochs for both the training and validation datasets. The graph indicates a rapid decline in loss, signifying efficient learning and convergence, with no signs of overfitting or instability.

FIG. 7B shows a confusion matrix, which provides a visualization of the model's classification accuracy. The confusion matrix confirms a high count of true positives and true negatives with minimal false positives and false negatives. This demonstrates the machine learning model's excellent precision and recall in predicting truncation events (e.g., truncation-prone regions).

FIG. 7C shows a table summarizing precision, recall, F1-score, and support values. Both the precision and recall scores approach 1.00, and the F-1 score is 0.98. This confirms the machine learning model's effectiveness in accurately predicting truncation events.

The robust machine learning model enables the identification of truncation-prone regions without having to manufacture the rAAV vector. By eliminating the reliance on costly post-production evaluations, the machine learning model significantly enhances the cost-effectiveness of vector development, while ensuring high vector homogeneity and stability (thereby reducing the likelihood of dysfunctional vectors), increased manufacturing efficiency (thereby minimizing failed production batches), and improved scalability to novel rAAV constructs (thereby allowing seamless adaption across different transgenes).

Beyond AAV vectors, the machine learning model serves as a generalizable machine learning framework for optimizing viral vector designs for gene therapy. In some embodiments, the machine learning model incorporates genomic and epigenetic features to further improve predictive powers.

### Additional Implementation Detail

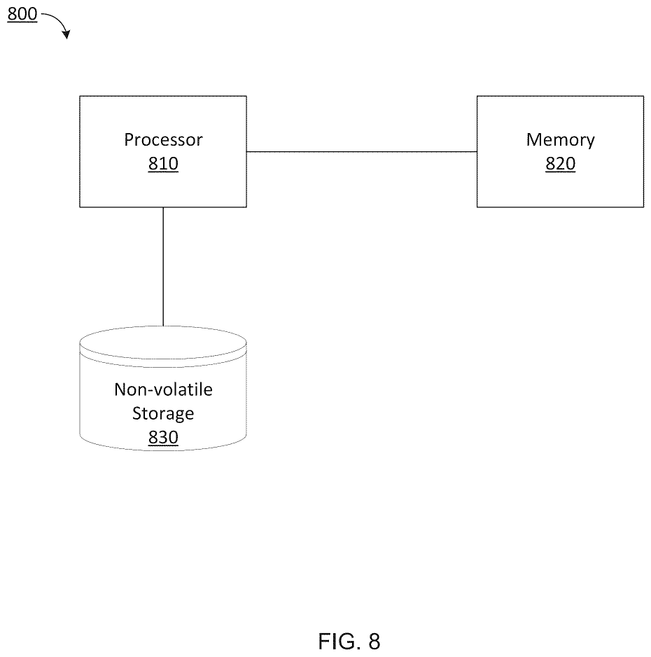

FIG. 8 is a block diagram of an illustrative computer system that may be used in implementing some embodiments of the technology described herein. An illustrative implementation of a computer system 800 that may be used in connection with any of the embodiments of the disclosure provided herein is shown in FIG. 8. The computer system 800 may include one or more processors 810 and one or more articles of manufacture that comprise non-transitory computer-readable storage media (e.g., memory 820 and one or more non-volatile storage media 830). The processor(s) 810 may control writing data to and reading data from the memory 820 and the non-volatile storage media 830 in any suitable manner, as the aspects of the technology described herein are not limited in this respect. To perform any of the functionality described herein, the processor(s) 810 may execute one or more processor-executable instructions stored in one or more non-transitory computer-readable storage media (e.g., the memory 820), which may serve as non-transitory computer-readable storage media storing processor-executable instructions for execution by the processor(s) 810.

The terms “program” or “software” are used herein in a generic sense to refer to any type of computer code or set of processor-executable instructions that can be employed to program a computer or other processor to implement various aspects of embodiments as described herein. Additionally, in some embodiments, one or more computer programs that when executed perform methods of the disclosure provided herein need not reside on a single computer or processor, but may be distributed in a modular fashion among different computers or processors to implement various aspects of the disclosure provided herein.

Processor-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in one or more non-transitory computer-readable storage media in any suitable form. For simplicity of illustration, data structures may be shown to have attributes that are related through location in the data structure. Such relationships may likewise be achieved by assigning storage for the attributes with locations in a non-transitory computer-readable medium that convey relationship between the attributes. However, any suitable mechanism may be used to establish relationships among information in attributes of a data structure, including through the use of pointers, tags or other mechanisms that establish relationships among data elements.

Also, various inventive concepts may be embodied as one or more processes. The acts performed as part of each process may be ordered in any suitable way. Accordingly, embodiments may be constructed in which acts are performed in an order different than illustrated, which may include performing some acts simultaneously, even though shown as sequential acts in illustrative embodiments.

### rAAV Production

Aspects of the disclosure relate to methods of identifying or selecting rAAV vectors that have reduced likelihood of truncation during replication and packaging relative to other rAAV vectors. Accordingly, in some aspects, the disclosure relates to isolated nucleic acids encoding rAAV vectors.

A “nucleic acid” sequence refers to a DNA or RNA sequence. In some embodiments, proteins and nucleic acids of the disclosure are isolated. As used herein, the term “isolated” means artificially produced. As used herein, with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.).

The isolated nucleic acids of the disclosure may be recombinant adeno-associated virus (AAV) vectors (rAAV vectors). In some embodiments, an isolated nucleic acid as described by the disclosure comprises adeno-associated virus (AAV) inverted terminal repeats (ITRs), or a variant thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, an expression cassette and its regulatory sequences, and 5′ and 3′ AAV inverted terminal repeats (ITRs). The isolated nucleic acid may comprise a region encoding, for example, a protein and/or an expression control sequence (e.g., a poly-A tail), as described elsewhere in the disclosure.

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of such a molecule employed in the disclosure is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. In some embodiments, the isolated nucleic acid further comprises a region (e.g., a second region, a third region, a fourth region, etc.) comprising a second AAV ITR. In some embodiments, an isolated nucleic acid encoding a transgene is flanked by AAV ITRs (e.g., in the orientation 5′-ITR-transgene-ITR-3′). In some embodiments, the AAV ITRs are AAV2 ITRs. In some embodiments, at least one of the AAV ITRs is a ΔITR, which lacks a terminal resolution site and induces formation of a self-complementary AAV (scAAV) vector. In some embodiments, the AAV ITRs are selected from the group consisting of AAV1 ITR, AAV2 ITR, AAV3 ITR, AAV4 ITR, AAV5 ITR, and AAV6 ITR.

Aspects of the disclosure relate to vectors comprising an isolated nucleic acid comprising a transgene. As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. In some embodiments, a vector is a viral vector, such as an rAAV vector, a lentiviral vector, an adenoviral vector, a retroviral vector, etc. Thus, the term includes cloning and expression vehicles, as well as viral vectors. In some embodiments, useful vectors are contemplated to be those vectors in which the nucleic acid segment to be transcribed is positioned under the transcriptional control of a promoter.

In some aspects, the disclosure provides isolated adeno-associated viruses (AAVs). As used herein with respect to AAVs, the term “isolated” refers to an AAV that has been artificially produced or obtained. Isolated AAVs may be produced using recombinant methods. Such AAVs are referred to herein as “recombinant AAVs.” Recombinant AAVs (rAAVs) preferably have tissue-specific targeting capabilities, such that a transgene of the rAAV will be delivered specifically to one or more predetermined tissue(s) (e.g., ocular tissues, neurons, liver, etc.). The AAV capsid is an important element in determining these tissue-specific targeting capabilities (e.g., tissue tropism). Thus, an rAAV having a capsid appropriate for the tissue being targeted can be selected.

Methods for obtaining recombinant AAVs having a desired capsid protein are well known in the art. (See, for example, US 2003/0138772), the contents of which are incorporated herein by reference in their entirety). Typically, the methods involve culturing a host cell which contains a nucleic acid sequence encoding an AAV capsid protein; a functional rep gene; a recombinant AAV vector composed of AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, an AAV capsid protein is of an AAV serotype selected from the group consisting of AAV1, AAV2, AAV3, AAV4, AAV5, AAV6, AAV7, AAV8, AAV9, AAV9.hr, AAVrh8, AAVrh10, AAVrh39, AAVrh43, AAV.PHP.B, AAVPHP.eB, and variants of any of the foregoing. In some embodiments, an AAV capsid protein is of a serotype derived from a non-human primate, for example AAVrh8 serotype. In some embodiments, the AAV capsid protein is an AAV8 capsid protein.

In some embodiments, an rAAV vector or rAAV particle comprises a mutant ITR that lacks a functional terminal resolution site (TRS). The term “lacking a terminal resolution site” can refer to an AAV ITR that comprises a mutation (e.g., a sense mutation such as a non-synonymous mutation, or missense mutation) that abrogates the function of the terminal resolution site (TRS) of the ITR, or to a truncated AAV ITR that lacks a nucleic acid sequence encoding a functional TRS (e.g., a ATRS ITR). Without wishing to be bound by any particular theory, a rAAV vector comprising an ITR lacking a functional TRS produces a self-complementary rAAV vector, for example as described by McCarthy (2008) Molecular Therapy 16(10):1648-1656.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

In some embodiments, the disclosure relates to a host cell containing a nucleic acid comprising a transgene. A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation. In some embodiments, the host cell is a mammalian cell, a yeast cell, a bacterial cell, an insect cell, a plant cell, or a fungal cell. In some embodiments, the host cell is a hepatocyte.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N. Y. Similarly, methods of generating rAAV virions are well known and the selection of a suitable method is not a limitation on the disclosure. See, e.g., K. Fisher et al., J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with an AAV vector (comprising a transgene flanked by ITR elements) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). Non-limiting examples of vectors suitable for use with the disclosure include pHLP 19, described in U.S. Pat. No. 6,001,650 and pRep 6 cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpes virus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

All definitions, as defined and used herein, should be understood to control over dictionary definitions, and/or ordinary meanings of the defined terms.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Use of ordinal terms such as “first,” “second,” “third,” etc., in the claims to modify a claim element does not by itself connote any priority, precedence, or order of one claim element over another or the temporal order in which acts of a method are performed. Such terms are used merely as terms to distinguish one claim element having a certain name from another element having a same name (but for use of the ordinal term).

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “substantially,” “approximately,” and “about” may be used to mean within ±20% of a target value in some embodiments, within ±10% of a target value in some embodiments, within +5% of a target value in some embodiments, within ±2% of a target value in some embodiments. The terms “approximately” and “about” may include the target value.

Having described several embodiments of the techniques described herein in detail, various modifications, and improvements will readily occur to those skilled in the art. Such modifications and improvements are intended to be within the spirit and scope of the disclosure. Accordingly, the foregoing description is by way of example only, and is not intended as limiting. The techniques are limited only as defined by the following claims and the equivalents thereto.

### Example

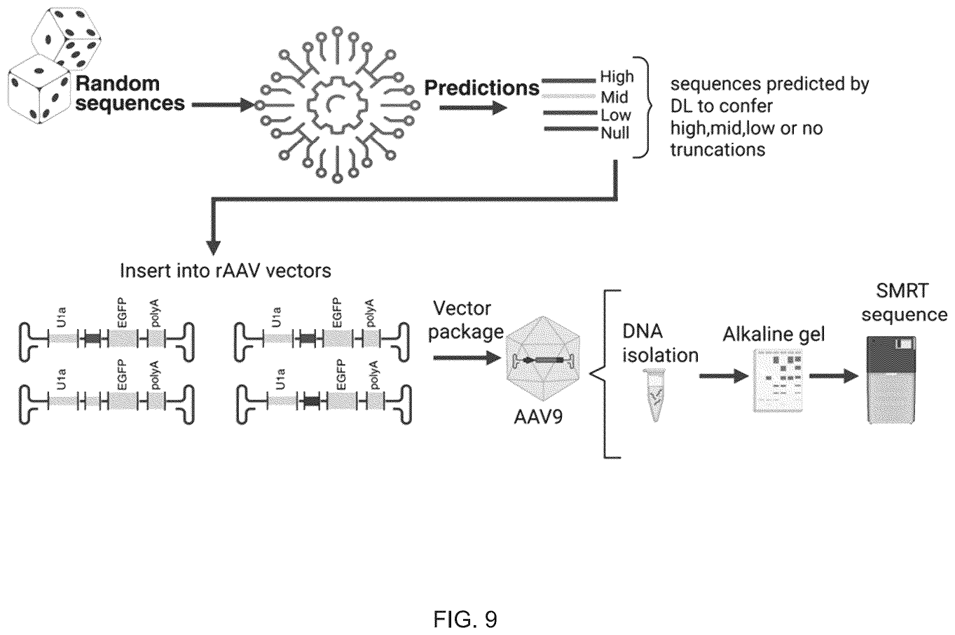

To directly test whether predicted truncation risk translates into experimental outcomes, large-scale in silico screening of approximately 10⁸ random 200 bp DNA sequences was performed using a trained truncation model as described by the disclosure. Sequences were ranked by predicted truncation risk and stratified into high, medium, low, and null categories. Representative sequences from each category were selected for experimental validation and inserted into a biologically relevant intronic region of a previously characterized rAAV construct identified by AAV-GPseq analysis (FIG. 9).

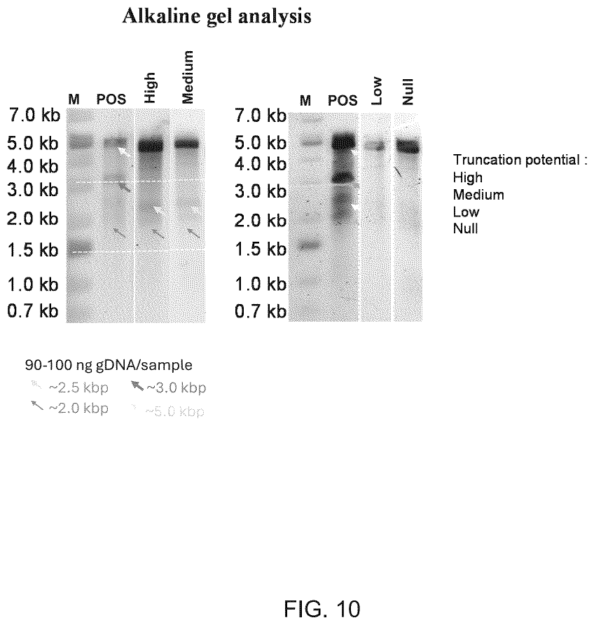

Alkaline gel electrophoresis of encapsidated vector genomes revealed a clear, graded truncation phenotype consistent with model predictions (FIG. 10). Constructs containing high- and medium-risk inserts displayed prominent truncated genome species in addition to full-length genomes, whereas low- and null-risk inserts predominantly yielded full-length genomes with minimal truncation. These results provided functional validation that predicted truncation risk correlates with observable genome integrity during rAAV production.

1. A method for using machine learning to predict truncation events in recombinant adeno-associated virus (rAAV) vectors, the method comprising:
using at least one computer hardware processor to perform:
obtaining rAAV vector sequence data; and
processing the rAAV vector sequence data using a trained neural network model to obtain an output indicating one or more positions in the rAAV vector sequence likely to be locations of truncation events,
wherein the trained neural network model comprises a series of convolutional layers followed by a recurrent layer configured to process output of the series of convolutional layers and to generate the output indicating the one or more positions in the rAAV vector sequence likely to be locations of truncation events.

2. The method of claim 1, wherein the series of convolutional layers comprises a series of one-dimensional (1D) convolutional layers.

3. The method of claim 2, wherein the series of 1D convolutional layers comprise filters of increasing depth.

4. The method of claim 1, wherein the series of 1D convolutional layers identify sequence motifs linked to truncation-prone regions of the rAAV vector sequence.

5. The method of claim 1, wherein the trained neural network model comprises maximum pooling layers within the series of convolutional layers.

6. The method of claim 1, wherein the recurrent layer comprises a series of gated recurrent units (GRUs).

7. The method of claim 6, wherein the series of GRUs comprises a series of bi-directional GRUs (Bi-GRUs).

8. The method of claim 1, wherein the recurrent layer comprises a long-short term memory (LSTM) layer.

9. The method of claim 1, wherein the trained neural network model further comprises a classification head configured to process output of the recurrent layer to generate output comprising: (1) an indication of whether or the rAAV vector sequence is truncation prone; and/or (2) an estimated expected frequency of truncation events for the rAAV vector sequence.

10. The method of claim 9, wherein the classification head comprises one or more fully-connected layers.

11. A method for selecting a particular rAAV vector sequence from a plurality of rAAV vector sequences, the method comprising:
performing the method of claim 1 on each of the plurality of rAAV vector sequences to obtain a respective plurality of outputs characterizing predicted truncation behavior of the plurality of rAAV vector sequences; and
selecting the particular rAAV vector sequence based on the plurality of outputs characterizing the predicted truncation behavior of the plurality of rAAV vector sequences.

12. The method of claim 11, further comprising synthesizing the particular rAAV vector sequence.

13. The method of claim 12, wherein the synthesizing comprises solid-phase oligonucleotide synthesis.

14. (canceled)

15. The method of claim 12, further comprising introducing the synthesized particular rAAV vector sequence into a mammalian cell.

16. The method of claim 15, wherein the mammalian cell is configured to express one or more AAV capsid proteins, one or more AAV Rep proteins, and/or one or more Adenoviral helper proteins.

17. (canceled)

18. The method of claim 1, wherein the rAAV vector sequence comprises two wild-type AAV inverted terminal repeats (ITRs).

19-21. (canceled)

22. A recombinant adeno-associated virus (rAAV) comprising:
(i) an rAAV vector comprising a sequence processed according to the method of claim 1; and
(ii) one or more AAV capsid proteins.

23. A composition comprising:
(i) a population of recombinant adeno-associated virus (rAAV) particles comprising less than 10% truncated rAAV vector genomes; and
(ii) mammalian cell culture media.

24. A system, comprising:
at least one computer hardware processor; and
at least one non-transitory computer-readable storage medium storing processor-executable instructions that, when executed by the at least one computer hardware processor, cause the at least one computer hardware processor to perform the method of claim 1.

25. At least one non-transitory computer-readable storage medium storing processor-executable instructions that, when executed by at least one computer hardware processor, cause the at least one computer hardware processor to perform the method of claim 1.
